(19) United States
(12) Patent Application Publication
(10) Pub. No.: US 2011/0269246 A1
(43) Pub. Date: Nov. 3, 2011

(54) MEASURING LEVELS OF FRATAXIN

(76) Inventors: Devin Oglesbee, Rochester, MN (US); Dietrich Matern, Rochester, MN (US); Grazia Isaya, Rochester, MN (US)

(21) Appl. No.: 13/143,304

(22) PCT Filed: Jan. 14, 2010
(86) PCT No.: PCT/US10/21067

§ 371 Date: Jul. 5, 2011

Related U.S. Application Data
(60) Provisional application No. 61/144,645, filed on Jan. 14, 2009.

(51) Int. Cl. G01N 33/566; G01N 33/82; G01N 21/64

(52) U.S. Cl. 436/501

(57) ABSTRACT

This document relates to methods and materials involved in measuring levels of a frataxin polypeptide present in a biological sample. For example, methods and materials related to the use of anti-frataxin antibody-bound microspheres and biotinylated anti-frataxin antibodies to measure the levels of a frataxin polypeptide in a biological sample from a mammal (e.g., a newborn human) are provided.

### CROSS-REFERENCE TO RELATED APPLICATIONS

This application claims the benefit of priority to U.S. Provisional Application Ser. No. 61/144,645, filed on Jan. 14, 2009. The disclosure of the prior application is considered part of (and is incorporated by reference in) the disclosure of this application.

### BACKGROUND

1. Technical Field

This document relates to methods and materials involved in measuring levels of a frataxin polypeptide present in a biological sample. For example, this document provides methods and materials related to the use of anti-frataxin antibody-bound microspheres and biotinylated anti-frataxin antibodies to measure the levels of a frataxin polypeptide in a biological sample from a mammal (e.g., a newborn human).

2. Background Information

Friedreich ataxia (FRDA), an autosomal recessive disease affecting 1:40,000 individuals, can be clinically characterized by progressive ataxia of all four limbs, cerebellar dysarthia, absent reflexes in the lower limbs, sensory loss, and pyramidal signs. Cardiomyopathy is found in most patients and represents the most frequent cause of premature death. The FRDA locus encodes a precursor polypeptide that is imported into the mitochondria and processed into a 155 amino acid polypeptide designated frataxin. Most patients are homozygous for a large GAA repeat expansion in the first intron of the FRDA gene, which impairs transcription, ultimately causing a severe reduction in the levels of frataxin.

### SUMMARY

This document relates to methods and materials involved in measuring levels of a frataxin polypeptide present in a biological sample. For example, methods and materials related to the use of anti-frataxin antibody-bound microspheres and biotinylated anti-frataxin antibodies to measure the levels of a frataxin polypeptide in a biological sample from a mammal (e.g., a newborn human) are provided. In some cases, the methods and materials provided herein can be used for diagnosis, identification of carrier status, and treatment monitoring of Friedreich ataxia patients and their families. In some cases, the methods and materials provided herein can be used to determine levels of a frataxin polypeptide at the same time as levels of other disease-associated polypeptides, for universal newborn screening for FRDA.

In general, this document features a method for assessing levels of a frataxin polypeptide in a mammal. The method comprises contacting an anti-frataxin antibody conjugated microsphere with a biological sample from a mammal, under conditions wherein a frataxin polypeptide present in the sample binds the microsphere, thereby forming a frataxin-microsphere complex, contacting the frataxin-microsphere complex with a detector-conjugated anti-frataxin antibody under conditions wherein the detector-conjugated anti-frataxin antibody binds the frataxin-microsphere complex, and quantifying the detector bound to the complex, thereby measuring levels of the frataxin polypeptide present in the sample. The mammal can be a human. The human can be a newborn. The biological sample can be a biological fluid. The biological fluid can be eluted from a dried blood sample. The dried blood sample can be on filter paper. The microsphere can comprise a fluorochrome. The microsphere can be carboxylated. The anti-frataxin antibody-conjugated microsphere can comprise a monoclonal antibody. The detector can be biotin. The quantification step can comprise contacting the complex with a composition comprising a streptavidin conjugate. The streptavidin conjugate can be streptavidin-R-phycoerythrin. The quantification step can comprise analysis on a flow cytometer.

In another aspect, this document features a method of assessing a human for Friedreich ataxia. The method comprises contacting an anti-frataxin antibody conjugated microsphere with a biological sample from a human, under conditions wherein a frataxin polypeptide present in the sample binds the microsphere, thereby forming a frataxin-microsphere complex, contacting the frataxin-microsphere complex with a detector-conjugated anti-frataxin antibody under conditions wherein the detector-conjugated anti-frataxin antibody binds the frataxin-microsphere complex, determining whether or not a biological fluid from the human contains a decreased level of a frataxin polypeptide, and communicating a diagnosis of Friedreich ataxia if a decreased level of a frataxin polypeptide is determined, thereby diagnosing the human with Friedreich ataxia. The human can be a newborn. The sample can be a dried blood sample. The microsphere can comprise a fluorochrome. The microsphere can be carboxylated. The anti-frataxin antibody-coupled microsphere can comprise a monoclonal antibody. The detector can be biotin. The quantification step can comprise contacting the complex with a composition comprising a streptavidin conjugate. The streptavidin conjugate can be streptavidin-R-phycoerythrin. The quantification step can comprise analysis on a flow cytometer.

In another aspect, this document features an article of manufacture. The article of manufacture comprises a vial containing anti-frataxin antibody-coupled microspheres, and a vial containing biotinylated anti-frataxin antibodies. The article of manufacture can comprise a vial containing purified human frataxin.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawing and the description below. Other features, objects, and advantages of the invention will be apparent from the description and drawings, and from the claims.

### DESCRIPTION OF DRAWINGS

FIG. 1 is a schematic representation of an exemplar microsphere-conjugated sandwich immunoassay for measurement of the level of a frataxin polypeptide present in a biological sample.

FIG. 2 is a graph plotting the amount of frataxin polypeptide (ng/punch) for an infant with FRDA and normal infants.

FIG. 3 is a graph plotting the amount of frataxin polypeptide (ng/punch) present in samples from 23 unaffected adults and 51 adults with FRDA.

FIG. 4 is a graph plotting the percent of ceruloplasmin polypeptide eluted from four newborn dried blood spots having less than one month of storage and five newborn dried-blood spots having greater than one year of storage.

### DETAILED DESCRIPTION

This document relates to methods and materials involved in measuring levels of a human frataxin polypeptide. For example, this document provides methods and materials related to the use of a capture sandwich immunoassay featuring anti-frataxin antibody-bound microspheres and anti-frataxin antibody-bound detection molecules, to measure the levels of a frataxin polypeptide present in a biological sample (e.g., a dried blood sample) from a mammal (e.g., a newborn human).

Any mammal can be assessed for reduced frataxin polypeptide levels using the methods and materials provided herein. For example, a human, mouse, cat, dog, or horse can be evaluated by assessing the levels a frataxin polypeptide in a biological sample to determine whether or not the mammal has FRDA. In some cases, a human suspected to have FRDA can be assessed. In some cases, a human between the ages of about 3-14 days old can be assessed. In some cases, a human older than about 14 days old can be assessed. In some cases, a human less than about 3 days old, e.g., a newborn infant, can be assessed.

The methods and materials described herein can be used to measure the levels of a frataxin polypeptide from a biological sample, such as a dried blood sample, biological fluid, or tissue. Examples of biological fluids include, without limitation, serum, plasma, and cerebrospinal fluid. A biological fluid can be obtained from a mammal by any appropriate method. For example, blood can be collected from heel or finger punctures by using single-use lancing devices.

The level of frataxin polypeptides can be detected using the methods and materials provided herein. For example, a capture-sandwich immunoassay can include using an anti-frataxin polypeptide antibody. An anti-frataxin polypeptide antibody can be labeled for detection. For example, an anti-frataxin polypeptide antibody can be labeled with a radioactive molecule, a fluorescent molecule, or a bioluminescent molecule. Frataxin polypeptides can also be detected indirectly using a labeled antibody that binds to an anti-frataxin polypeptide antibody that binds to a frataxin polypeptide. An anti-frataxin polypeptide antibody can bind to a frataxin polypeptide with an affinity of at least 10⁴ mol⁻¹ (e.g., at least 10⁵, 10⁶, 10⁷, 10⁸, 10⁹, 10¹⁰, 10¹¹ or 10¹² mol⁻¹). An anti-frataxin antibody can be a polyclonal or monoclonal antibody. For example, monoclonal anti-human frataxin polypeptide antibodies are commercially available, e.g., from MitoSciences Inc., clone #17A11AC7.

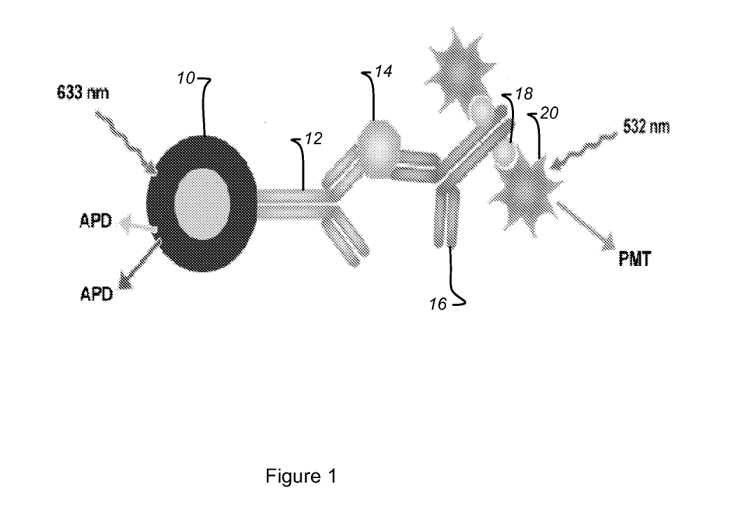

The methods and materials described herein can be used in a sandwich-capture immunoassay. Referring to FIG. 1, a frataxin polypeptide 14 can be sandwiched by a microsphere 10 conjugated to an antibody 12, and detector-conjugated antibody 16. A detector 18 can bind a reporter compound 20 to produce a signal that can be detected and quantified. In some cases, a microsphere can be conjugated to a frataxin polypeptide-specific antibody. Any appropriate method can be used to conjugate a microsphere to an anti-frataxin antibody, e.g., incubation. Conditions for antibody conjugation can be varied depending on the affinity of the antibody for a frataxin polypeptide, the optimal antibody concentration on the microsphere, and the surface chemistry of the microsphere. In some cases, a microsphere can be coated to reduce non-specific binding of serum proteins, e.g., a carboxylated microsphere. In some cases, a microsphere can have defined spectral properties. For example, a microsphere can emit fluorescence in response to laser line excitation.

Microsphere bound-frataxin polypeptide can be quantified indirectly using a detector-conjugated antibody. For example, an anti-frataxin antibody can be used as a detector antibody, such as polyclonal antibody HFxn Ab2518. The detector antibody can be labeled with a radioactive molecule, a fluorescent molecule, a bioluminescent molecule, an enzyme, or a ligand. For example, a detector antibody can be biotinylated, and levels of frataxin polypeptides in a biological sample can be measured by detecting fluorescence from an avidin conjugate, e.g., streptavidin-R-phycoerythrin.

For example, the concentration of frataxin polypeptides can be assessed by any instrument capable of analyzing a solid phase sandwich immunoassay. In some cases, captured frataxin polypeptides can be measured by instruments capable of quantifying a detector-conjugated antibody, e.g., flow cytometer. For example, materials described herein can be analyzed by a flow cytometer such as a Luminex 100™ or 200™ instrument.

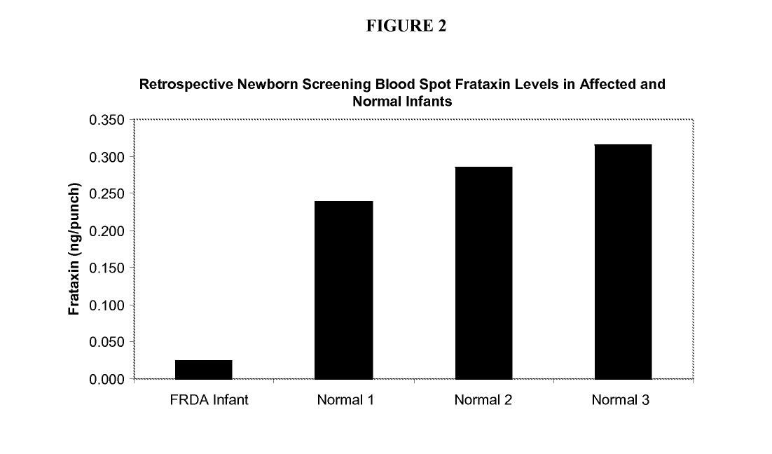

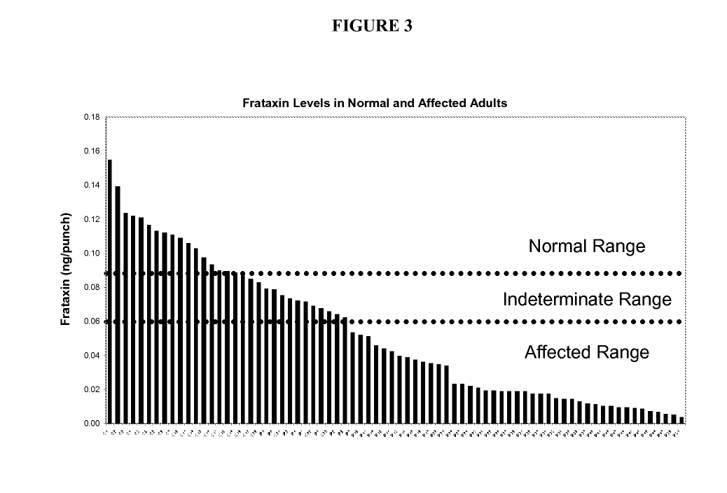

Once the level of a frataxin polypeptide in a biological fluid from a mammal is determined, then the level can be compared to a median level or a cutoff level and used to determine whether or not the mammal has FRDA. If it is determined that a biological fluid from a mammal contains a reduced or decreased level of a frataxin polypeptide, then the mammal can be classified as having FRDA. For example, if it is determined that a biological fluid from a human infant (e.g., dried-blood spot sample) contains less than 0.2 (e.g., less than 0.15, less than 0.1, or less than 0.05) ng of a frataxin polypeptide per punch, then the human infant can be classified as having FRDA. See, e.g., FIG. 2. In some cases, if it is determined that a biological fluid from a human adult (e.g., dried-blood spot sample) contains less than 0.06 (e.g., less than 0.05, less than 0.04, or less than 0.03) ng of a frataxin polypeptide per punch, then the human adult can be classified as having FRDA. See, e.g., FIG. 3.

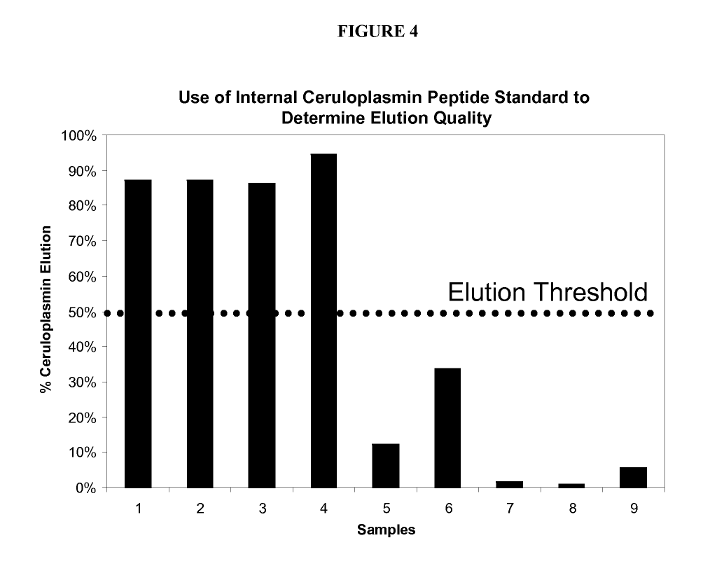

In some cases, the level of a frataxin polypeptide in a biological fluid can be used in combination with one or more other factors to determine whether or not a mammal has FRDA. For example, a frataxin polypeptide level in a biological fluid can be used in combination with a gait or reflex test. In some cases, assessing the level of a frataxin polypeptide in a biological fluid (e.g., dried-blood spot sample) can include using the level of an internal reference polypeptide (e.g., ceruloplasmin polypeptides) to confirm the quality of the sample. See, e.g., FIG. 4. For example, if the elution of an internal reference polypeptide is below a permissible threshold level, then the quality of the frataxin polypeptide measurement can be insufficient to report and an additional sample or specimen may be needed for analysis.

The term “decreased level” as used herein with respect to the level of a frataxin polypeptide is any level that is below a threshold level or a median frataxin polypeptide level in a biological fluid (e.g., dried blood sample) from a random population of mammals (e.g., a random population of 10, 20, 30, 40, 50, 100, or 500 mammals) that do not have FRDA. In some cases, a decreased level of a frataxin polypeptide can be an undetectable level of a frataxin polypeptide in biological sample.

In some cases, as determined in dried blood spots, a level of a frataxin polypeptide that is greater than 0.08 ng/punch can be considered normal, a level of a frataxin polypeptide that is between 0.08 and 0.06 ng/punch can be considered intermediate, and a level of a frataxin polypeptide that is less than 0.06 ng/punch can be considered a decreased level. A mammal (e.g., a human infant or adult) having such a decreased level can be classified as having FRDA.

This document also provides methods and materials to assist medical or research professionals in determining whether or not a mammal has FRDA. Medical professionals can be, for example, doctors, nurses, medical laboratory technologists, and pharmacists. Research professionals can be, for example, principal investigators, research technicians, postdoctoral trainees, and graduate students. A professional can be assisted by (1) determining the level of a frataxin polypeptide in a biological fluid, and (2) communicating information about the level to that professional.

After the level of a frataxin polypeptide is reported, a medical professional can take one or more actions that can affect patient care. For example, a medical professional can record the level of a frataxin polypeptide in a patient's medical record. In some cases, a medical professional can record a diagnosis of FRDA, or otherwise transform the patient's medical record, to reflect the patient's medical condition. In some cases, a medical professional can review and evaluate a patient's entire medical record, and assess multiple treatment strategies, for clinical intervention of a patient's condition.

A medical professional can initiate or modify treatment for FRDA symptoms after receiving information regarding a patient's frataxin polypeptide levels. In some cases, a medical professional can compare previous reports of frataxin polypeptide levels with the recently communicated frataxin polypeptide level, and recommend a change in therapy. In some cases, a medical professional can enroll a patient in a clinical trial for novel therapeutic intervention of FRDA symptoms. In some cases, a medical professional can elect waiting to begin therapy until the patient's symptoms require clinical intervention.

A medical professional can communicate the levels of a frataxin polypeptide to a patient or a patient's family. In some cases, a medical professional can provide a patient and/or a patient's family with information regarding FRDA, including treatment options, prognosis, and referrals to specialists, e.g., cardiologists and/or genetic counselors. In some cases, a medical professional can provide a copy of a patient's medical records to communicate the levels of a frataxin polypeptide, to a specialist.

A research professional can apply information regarding a subject's frataxin polypeptide levels to advance FRDA research. For example, a researcher can compile data on frataxin polypeptide levels with information regarding the efficacy of a drug for treatment of FRDA symptoms to identify an effective treatment. In some cases, a research professional can obtain a subject's frataxin polypeptide levels to evaluate a subject's enrollment, or continued participation in a research study or clinical trial. In some cases, a research professional can classify the severity of a subject's condition, based on the levels of a frataxin polypeptide. In some cases, a research professional can communicate a subject's frataxin polypeptide level to a medical professional. In some cases, a research professional can refer a subject to a medical professional for clinical assessment of FRDA, and treatment of FRDA symptoms.

Any appropriate method can be used to communicate information to another person (e.g., a professional). For example, information can be given directly or indirectly to a professional. For example, a laboratory technician can input frataxin polypeptide levels into a computer-based record. In some cases, information is communicated by making an physical alteration to medical or research records. For example, a medical professional can make a permanent notation or flag a medical record for communicating a diagnosis to other medical professionals reviewing the record. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

The invention will be further described in the following example, which does not limit the scope of the invention described in the claims.

### EXAMPLE

### Example 1

### Quantification of Frataxin in Dried Blood Samples

### Sample Collection

Whole blood is applied on filter paper card (grade 903) in 100 μL drops, using EDTA-free devices or capillary tubes. The paper cards are dried at room temperature, in a horizontal position for three or more hours. Specimens are stored at ambient, refrigerated or frozen conditions, but not for more than 48 hours at temperatures exceeding 25° C.

### Sample Preparation

Two 3 mm (⅛″) blood samples are punched from each control, one sample from a blank filter paper, and one blood sample from each patient into a flat bottom 96-well plate, as shown in Table 1. Each assay is run with a Low and Normal control.

TABLE 1

| | 1 | 2 | 3 | 4 | 5 | 6 | 7 | 8 | 9 | 10 | 11 | 12 |
|---|---|---|---|---|---|---|---|---|---|---|---|---|
| A | Std | Normal | Pt 7 | Pt 15 | Pt 23 | Pt 31 | Pt 39 | Pt 47 | Pt 55 | Pt 63 | Pt 71 | Pt 79 |
| | 4.4 | control | punch 1 | punch 1 | punch 1 | punch 2 | punch 1 | punch 2 | punch 1 | punch 2 | punch 1 | punch 1 |
| B | Std | Low | Pt 8 | Pt 16 | Pt 24 | Pt 32 | Pt 40 | Pt 48 | Pt 56 | Pt 64 | Pt 72 | Pt 80 |
| | 2.2 | Control | punch 1 | punch 1 | punch 1 | punch 2 | punch 1 | punch 2 | punch 1 | punch 2 | punch 1 | punch 1 |
| C | Std | Pt 1 | Pt 9 | Pt 17 | Pt 25 | Pt 33 | Pt 41 | Pt 49 | Pt 57 | Pt 65 | Pt 73 | Pt 81 |
| | 1.1 | punch 1 | punch 1 | punch 1 | punch 1 | punch 1 | punch 1 | punch 2 | punch 1 | punch 2 | punch 1 | punch 1 |
| D | Std | Pt 2 | Pt 10 | Pt 18 | Pt 26 | Pt 34 | Pt 42 | Pt 50 | Pt 58 | Pt 66 | Pt 74 | Pt 82 |
| | 0.55 | punch 1 | punch 1 | punch 1 | punch 1 | punch 1 | punch 1 | punch 2 | punch 1 | punch 2 | punch 1 | punch 1 |
| E | Std | Pt 3 | Pt 11 | Pt 19 | Pt 27 | Pt 35 | Pt 43 | Pt 51 | Pt 59 | Pt 67 | Pt 75 | Pt 83 |
| | 0.28 | punch 1 | punch 1 | punch 1 | punch 1 | punch 1 | punch 1 | punch 2 | punch 1 | punch 2 | punch 1 | punch 1 |
| F | Std | Pt 4 | Pt 12 | Pt 20 | Pt 28 | Pt 36 | Pt 44 | Pt 52 | Pt 60 | Pt 68 | Pt 76 | Pt 84 |
| | 0.14 | punch 1 | punch 1 | punch 1 | punch 1 | punch 2 | punch 1 | punch 2 | punch 1 | punch 2 | punch 1 | punch 1 |
| G | Std | Pt 5 | Pt 13 | Pt 21 | Pt 29 | Pt 37 | Pt 45 | Pt 53 | Pt 61 | Pt 69 | Pt 77 | Normal |
| | 0.007 | punch 1 | punch 1 | punch 1 | punch 1 | punch 2 | punch 1 | punch 2 | punch 1 | punch 2 | punch 1 | control |
| H | Blank | Pt 6 | Pt 14 | Pt 22 | Pt 30 | Pt 38 | Pt 46 | Pt 54 | Pt 62 | Pt 70 | Pt 78 | Low |
| | | punch 1 | punch 1 | punch 1 | punch 1 | punch 2 | punch 1 | punch 2 | punch 1 | punch 2 | punch 1 | Control |

### Calibration

A calibration curve is generated with each analytical batch and is found in column 1 on the plate. A 7-point calibration curve is generated and analyzed (Table 2). The calibration is acceptable if the standard curve of the dilutions has R²>0.9900.

TABLE 2

| Plate wells used | Concentration of |
|---|---|
| for calibration | calibrators ng/well |
| A1 | 4.4 |
| B1 | 2.2 |
| C1 | 1.1 |
| D1 | 0.55 |
| E1 | 0.28 |
| F1 | 0.14 |
| G1 | 0.007 |
| H1 | 0 |

### Microsphere Capture-Sandwich Immunoassay

Into each well of a microwell plate, 250 μL of assay buffer (filtered PBS, 1% BSA, pH 7.4, 0.02% Sodium Azide) is added. Column 1 is reserved for the standards. The first two wells and last two wells on the plate are reserved for the controls. The plate is covered with an EZ Seal plate sealer and placed on a MaxQ® orbital shaking incubator at 37° C. for 3 hours. Frataxin antibody-coupled microspheres (Carboxylated Microspheres Region # 8, Luminex, Inc; clone 17A11AC7, MitoSciences Inc.) are suspended by vortex and sonication for about 20 seconds, and diluted to a final concentration of 4000 microspheres/50 μL in assay buffer. A 1.2 μm Millipore filter plate is pre-wetted with 100 μL/well of assay buffer aspirated by vacuum manifold. A 50 μL aliquot of the microsphere mixture is pipetted into the appropriate wells of the filter plate. 50 μL aliquots of the standards and controls are pipetted into the appropriate wells. 50 μL is transferred from all eluted patient wells to the appropriate wells on a 96-well Millipore filter plate. The reactions are mixed gently by pipetting up and down several times with a multi-channel pipettor. The filter plate is covered and incubated for 90 minutes at 37° C. at 225 rpm on the Barnstead MaxQ® orbital shaking incubator.

After incubation, the supernatant is aspirated by vacuum manifold. Each well is washed twice with 100 μL of assay buffer and aspirated by vacuum manifold. The microspheres are resuspended in 50 μL of assay buffer by gently pipetting up and down five times with a multi-channel pipettor. The HFxn Ab2518 antibodies are biotinylated for use as detection antibodies (EZ Link Micro Sulfo-NHS-LC-Biotinylation Kit, Pearce, Inc.). 50 μL of the diluted biotinylated detection antibody are added to each well, and mixed gently. The filter plate is covered and incubated for 90 minutes at 37° C. at 225 rpm on the Barnstead MaxQ® orbital shaking incubator. The supernatant is aspirated by vacuum manifold. Each well is washed twice with 100 μL of assay buffer and aspirated by vacuum manifold. The microspheres are resuspended in 50 μL of assay buffer, and 50 μL streptavidin-R-phycoerythrin (SAPE) (4 μg/mL) is added to each well. The filter plate is covered and incubated for 30 minutes at room temperature on a plate shaker. The supernatant is aspirated by vacuum manifold. Each well is washed twice with 100 μL of assay buffer and aspirated by vacuum manifold.

After washing and resuspending in 100 μL of assay buffer, 50-75 μL of the microsphere solution is analyzed on the Luminex® analyzer according to the system manual. Briefly, microspheres are excited by a 633 nm laser, and emit fluorescence that is detected by two avalanche photo diodes (APD). The analyte reporter (streptavidin-R-phycoerthrin) is excited by a 532 nm laser, and fluorescence is detected by a photomultiplier tube (PMT)) (FIG. 1).

### Results

By monitoring the spectral properties of the beads and the amount of associated SAPE fluorescence, the concentration of frataxin polypeptides in each well is determined. These data are correlated to the concentration of the calibrator. The concentration of the calibrator is converted from ng/well to the final reporting units of ng/μL blood. Results below the normal range are called back to the requesting physician by the Genetic Counselor or consultant on-call.

### Example 2

### Quantification of Frataxin in Dried Blood Samples

### Sample Collection

Whole blood is applied on filter paper card (grade 903) either in 50 μL drops, using EDTA-free devices or dabbed onto the card if the blood is obtained from a heel or fingerstick. The paper cards are dried at room temperature, in a horizontal position for three or more hours. Specimens are stored at ambient, refrigerated or frozen conditions, but not for more than 48 hours at temperatures exceeding 25° C.

### Sample Preparation

Two 3 mm (⅛″) blood samples are punched from each control, one sample from a blank filter paper, and one blood sample from each patient into a flat bottom 96-well plate, as shown in Table 3. Each assay is run with a Low and Normal control.

TABLE 3

| | 1 | 2 | 3 | 4 | 5 | 6 | 7 | 8 | 9 | 10 | 11 | 12 |
|---|---|---|---|---|---|---|---|---|---|---|---|---|
| A | Std | Normal | Pt 7 | Pt 15 | Pt 23 | Pt 31 | Pt 39 | Pt 47 | Pt 55 | Pt 63 | Pt 71 | Pt 79 |
| | 0.88 | control | punch 1 | punch 1 | punch 1 | punch 2 | punch 1 | punch 2 | punch 1 | punch 2 | punch 1 | punch 1 |
| B | Std | Low | Pt 8 | Pt 16 | Pt 24 | Pt 32 | Pt 40 | Pt 48 | Pt 56 | Pt 64 | Pt 72 | Pt 80 |
| | 0.44 | Control | punch 1 | punch 1 | punch 1 | punch 2 | punch 1 | punch 2 | punch 1 | punch 2 | punch 1 | punch 1 |
| C | Std | Pt 1 | Pt 9 | Pt 17 | Pt 25 | Pt 33 | Pt 41 | Pt 49 | Pt 57 | Pt 65 | Pt 73 | PT 81 |
| | 0.22 | punch 1 | punch 1 | punch 1 | punch 1 | punch 1 | punch 1 | punch 2 | punch 1 | punch 2 | punch 1 | punch 1 |
| D | Std | Pt 2 | Pt 10 | Pt 18 | Pt 26 | Pt 34 | Pt 42 | Pt 50 | Pt 58 | Pt 66 | Pt 74 | Pt 82 |
| | 0.11 | punch 1 | punch 1 | punch 1 | punch 1 | punch 1 | punch 1 | punch 2 | punch 1 | punch 2 | punch 1 | punch 1 |
| E | Std | Pt 3 | Pt 11 | Pt 19 | Pt 27 | Pt 35 | Pt 43 | Pt 51 | Pt 59 | Pt 67 | Pt 75 | Pt 83 |
| | 0.05 | punch 1 | punch 1 | punch 1 | punch 1 | punch 1 | punch 1 | punch 2 | punch 1 | punch 2 | punch 1 | punch 1 |
| F | Std | Pt 4 | Pt 12 | Pt 20 | Pt 28 | Pt 36 | Pt 44 | Pt 52 | Pt 60 | Pt 68 | Pt 76 | Pt 84 |
| | 0.03 | punch 1 | punch 1 | punch 1 | punch 1 | punch 2 | punch 1 | punch 2 | punch 1 | punch 2 | punch 1 | punch 1 |
| G | Std | Pt 5 | Pt 13 | Pt 21 | Pt 29 | Pt 37 | Pt 45 | Pt 53 | Pt 61 | Pt 69 | Pt 77 | Normal |
| | 0.01 | punch 1 | punch 1 | punch 1 | punch 1 | punch 2 | punch 1 | punch 2 | punch 1 | punch 2 | punch 1 | control |
| H | Blank | Pt 6 | Pt 14 | Pt 22 | Pt 30 | Pt 38 | Pt 46 | Pt 54 | Pt 62 | Pt 70 | Pt 78 | Low |
| | | punch 1 | punch 1 | punch 1 | punch 1 | punch 2 | punch 1 | punch 2 | punch 1 | punch 2 | punch 1 | Control |

### Calibration

A calibration curve is generated with each analytical batch and is found in column 1 on the plate. Calibrators are prepared from purified human Ceruloplasmin (CP11, Mayo Clinic) and purified recombinant human Frataxin (#183, Mayo Clinic). A 7-point calibration curve is generated and analyzed (Table 4). The calibration is acceptable if the standard curve of the dilutions has R²>0.9900.

TABLE 4

| Plate wells used | Concentration of |
|---|---|
| for calibration | calibrators ng/well |
| A1 | 0.88 |
| B1 | 0.44 |
| C1 | 0.22 |
| D1 | 0.11 |
| E1 | 0.05 |
| F1 | 0.03 |
| G1 | 0.01 |
| H1 | 0 |

### Microsphere Capture-Sandwich Immunoassay

Into each well of a microwell plate, 200 μL of assay buffer (filtered PBS, 1% BSA, pH 7.4, 0.02% Sodium Azide) is added. Column 1 is reserved for the standards. The first two wells and last two wells on the plate are reserved for the controls. The plate is covered and placed on an orbital shaker at ambient temperature for 3 hours. Anti-frataxin antibody-coupled microspheres (Carboxylated Microspheres Region # 11, Luminex, Inc; antibody clone 17A11AC7, MitoSciences Inc.) and anti-Ceruloplasmin antibody-coupled microspheres (Carboxylated Microspheres Region # 8, Luminex, Inc; antibody clone CR6010RP, Cortex BioChem) are suspended by vortex and sonication for about 20 seconds, and diluted to a final concentration of 3000 microspheres/50 μL in assay buffer. A 1.2 μm Millipore filter plate is pre-wetted with 100 μL/well of assay buffer aspirated by vacuum manifold. A 50 μL aliquot of the microsphere mixture is pipetted into the appropriate wells of the filter plate. 50 μL aliquots of the standards and controls are pipetted into the appropriate wells. 50 μL is transferred from all eluted patient wells to the appropriate wells on a 96-well Millipore filter plate. The reactions are mixed gently by pipetting up and down several times with a multi-channel pipettor. The filter plate is covered and incubated for 90 minutes at ambient temperature at >200 rpm on the orbital shaker.

After incubation, the supernatant is aspirated by vacuum manifold. Each well is washed three times with 100 μL of wash buffer (filtered PBS, 0.05& Tween 20, pH 7.4) and aspirated by vacuum manifold. The microspheres are resuspended in 50 μL of assay buffer by gently pipetting up and down five times with a multi-channel pipettor. Anti-Frataxin antibodies (Clone HFxn Ab2518, Mayo Clinic) and anti-ceruloplasmin antibodies (clone WD 1.1, Mayo Clinic) are biotinylated for use as detection antibodies (EZ Link Micro Sulfo-NHS-LC-Biotinylation Kit, Pearce, Inc.). 50 μL of the combined diluted biotinylated detection antibody are added to each well, and mixed gently. The filter plate is covered and incubated for 90 minutes at ambient at >200 rpm on the orbital shaker. The supernatant is aspirated by vacuum manifold. Each well is washed twice with 100 μL of wash buffer and aspirated by vacuum manifold. The microspheres are resuspended in 50 μL of assay buffer, and 50 μL streptavidin-R-phycoerythrin (SAPE) (1 μg/mL) is added to each well. The filter plate is covered and incubated for 30 minutes at room temperature on a plate shaker. The supernatant is aspirated by vacuum manifold. Each well is washed twice with 100 μL of wash buffer and aspirated by vacuum manifold.

After washing and resuspending in 100 μL of assay buffer, 80 μL of the microsphere solution is analyzed on the Luminex analyzer according to the system manual. Briefly, microspheres are excited by a 633 nm laser, and emit fluorescence that is detected by two avalanche photo diodes (APD). The analyte reporter (streptavidin-R-phycoerthrin) is excited by a 532 nm laser, and fluorescence is detected by a photomultiplier tube (PMT)) (FIG. 1).

### Results

By monitoring the spectral properties of the beads and the amount of associated SAPE fluorescence, the concentration of frataxin polypeptides in each well is determined. These data are correlated to the concentration of the calibrator. The concentration of the calibrator is converted from ng/well to the final reporting units of ng/μL of blood or ng/punch. The co-analysis of the protein ceruloplasmin, provides an additional quality control parameter to evaluate the elution of the proteins from the blood spots. Low Frataxin results combined with low Ceruloplasmin results would indicate poor elution or improper handling of the blood spot rather than a below normal Frataxin result. Frataxin results below the normal range are called back to the requesting physician by the Genetic Counselor or consultant on-call.

### Example 3

### Identifying Infants Having FRDA

A single retrospective, newborn screening dried-blood spot was obtained with consent from an affected FRDA infant and was processed, along side three different retrospective newborn screening dried-blood spots obtained from unaffected infants (samples: Normal 1, 2, and 3), using a frataxin quantification, dried blood spot assay.

The quantity of frataxin polypeptide (ng) per 3 mm blood-spot-punch is shown in FIG. 2. Theses results demonstrate that the Frataxin Quantification, Dried Blood Spot assay can be used to detect and quantify the amount of frataxin polypeptide in a newborn dried-blood spot. The amount of frataxin polypeptide in a pre-symptomatic Friedreich ataxia infant was 5-times less than normal controls, demonstrating that this assay can be used to screen for pre-symptomatic Friedreich ataxia individuals in the newborn period.

### Example 4

### Identifying Adults with FRDA

23 unaffected participant samples and 51 Friedreich ataxia participant samples were analyzed by the Frataxin Quantification, Dried-Blood Spot assay. The amount of frataxin measured in each 3 mm dried-blood spot punch is provided in FIG. 3.

These results demonstrate that samples from Friedreich ataxia individuals have the lowest levels of frataxin measured (<0.06 ng/punch). There was minimal overlap in frataxin levels between normals and affected individuals. Frataxin levels between 0.08-0.06 ng/punch occurred for a minor number of normal and affected individuals, and thus, indicated that there is a range of frataxin polypeptide levels in dried-blood spots. A “normal range” for frataxin polypeptide levels was found at >0.06 ng/punch, and the majority of FRDA patients contained frataxin polypeptide levels below this amount.

### Example 5

### Additional Measurements to Confirm Decreased Frataxin Polypeptide Levels

Four newborn dried blood spots (<1 month of storage) and 5 newborn dried-blood spots (>1 year of storage) were processed with the Frataxin Quantification, Dried-Blood Spot assay. The percent of ceruloplasmin polypeptide detected as compared to a co-run control was plotted for each sample (FIG. 4).

These results demonstrate that the percent of ceruloplasmin elution from a dried-blood spot can be helpful for determining the quality of protein elution. In dried-blood spot samples with low levels of frataxin, the percent of ceruloplasmin elution can be determined in order to identify whether the low frataxin level is specific (i.e., as expected for a FRDA patient), or non-specific as observed in compromised dried-blood spot samples (e.g., samples stored for >1 year).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for assessing levels of a frataxin polypeptide in a mammal, wherein said method comprises
(a) contacting an anti-frataxin antibody conjugated microsphere with a biological sample from a mammal, under conditions wherein a frataxin polypeptide present in said sample binds said microsphere, thereby forming a frataxin-microsphere complex,
(b) contacting said frataxin-microsphere complex with a detector-conjugated anti-frataxin antibody under conditions wherein said detector-conjugated anti-frataxin antibody binds said frataxin-microsphere complex, and
(c) quantifying said detector bound to said complex, thereby measuring levels of said frataxin polypeptide present in said sample.

2. The method of claim 1, wherein said mammal is a human.

3. The method of claim 2, wherein said human is a newborn.

4. The method of claim 1, wherein said biological sample is a biological fluid.

5. The method of claim 4, wherein said biological fluid is eluted from a dried blood sample.

6. The method of claim 5, wherein said dried blood sample is on filter paper.

7. The method of claim 1, wherein said microsphere comprises a fluorochrome.

8. (canceled)

9. The method of claim 1, wherein said anti-frataxin antibody-conjugated microsphere comprises a monoclonal antibody.

10. The method of claim 1, wherein said detector is biotin.

11. The method of claim 1, wherein said quantification step comprises contacting said complex with a composition comprising a streptavidin conjugate.

12. (canceled)

13. The method of claim 1, wherein said quantification step comprises analysis on a flow cytometer.

14. A method of assessing a human for Friedreich ataxia, wherein said method comprises
(a) contacting an anti-frataxin antibody conjugated microsphere with a biological sample from a human, under conditions wherein a frataxin polypeptide present in said sample binds said microsphere, thereby forming a frataxin-microsphere complex,
(b) contacting said frataxin-microsphere complex with a detector-conjugated anti-frataxin antibody under conditions wherein said detector-conjugated anti-frataxin antibody binds said frataxin-microsphere complex,
(c) determining whether or not a biological fluid from said human contains a decreased level of a frataxin polypeptide, and
(d) communicating a diagnosis of Friedreich ataxia if a decreased level of a frataxin polypeptide is determined, thereby diagnosing said human with Friedreich ataxia.

15. The method of claim 14, wherein said human is a newborn.

16. The method of claim 15, wherein said sample is a dried blood sample.

17. The method of claim 14, wherein said microsphere comprises a fluorochrome.

18-19. (canceled)

20. The method of claim 14, wherein said detector is biotin.

21. The method of claim 14, wherein said quantification step comprises contacting said complex with a composition comprising a streptavidin conjugate.

22. (canceled)

23. The method of claim 14, wherein said quantification step comprises analysis on a flow cytometer.

24. An article of manufacture comprising a vial containing anti-frataxin antibody-coupled microspheres, and a vial containing biotinylated anti-frataxin antibodies.

25. The article of manufacture of claim 24, comprising a vial containing purified human frataxin.
